(19) United States
(12) Patent Application Publication
(10) Pub. No.: US 2005/0009811 A1
(43) Pub. Date: Jan. 13, 2005

(54) Saquinavir mesylate oral dosage form

(76) Inventors: Antonio A. Albano, Clifton, NJ (US); Martin Howard Infeld, Upper Montclair, NJ (US); Wantanee Phuapradit, Montville, NJ (US); Navnit Hargovindas Shah, Clifton, NJ (US); Lin Zhang, Bloomfield, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 10/886,765

(22) Filed: Jul. 8, 2004

Related U.S. Application Data
(60) Provisional application No. 60/568,204, filed on May 5, 2004.
(60) Provisional application No. 60/486,600, filed on Jul. 11, 2003.

(51) Int. Cl. A61K 31/551

(52) U.S. Cl. 514/220

(57) ABSTRACT

A solid unit oral pharmaceutical dosage form of saquinavir mesylate is provided comprising micronized saquinavir mesylate in an amount of from 250 mg to 800 mg calculated as free base, and a pharmaceutically acceptable binder, disintegrant, and water soluble carrier. A solid unit dosage form of saquinavir mesylate is provided comprising from 60% to 80% micronized saquinavir mesylate based on the mesylate salt, 4% to 8% water soluble binder, a disintegrant and a carrier, wherein each percentage is of the kernel weight.

This application claims the benefit of Provisional Application(s) Ser. No. 60/568,204, filed May 5, 2004 and Ser. No. 60/486,600, filed Jul. 11, 2003.

### BACKGROUND OF THE INVENTION

Saquinavir mesylate is one of several protease inhibitors used to limit viral replication and improve immune function in HIV-infected individuals. Saquinavir mesylate is commercially available as a 200 mg capsule (calculated as Saquinavir free base). It is sold under the name INVIRASE® by Hoffmann-La Roche, Inc., and is indicated for use in combination with an antiretroviral nucleoside analogue for the treatment of advanced human immunodeficiency virus (HIV) infection in select patients.

Saquinavir mesylate is a white to off-white, very fine crystalline powder having a molecular weight of 766.96. The molecular weight of the free base is 670.86. The drug is highly hydrophobic. INVIRASE® (Saquinavir Mesylate) 200 mg capsules have low oral bioavailability, which is thought to be due to its incomplete absorption and extensive first pass metabolism. [Physician's Desk Reference, 57^{th} Ed. (2003).] Saquinavir mesylate has very low aqueous solubility (i.e. 2.2 mg/mL in water, 0.08 mg/mL in simulated gastric fluid and is practically insoluble in simulated intestinal fluid at 25° C.). In addition, the drug exhibits pH-dependent solubility characteristics, having limited solubility in simulated gastric fluid while being practically insoluble in simulated intestinal fluid. Absolute bioavailability averaged 4% (CV 73%, range: 1-9%) in 8 healthy volunteers who received a single 600 mg dose (3×200 mg capsules) of saquinavir following a high fat breakfast. Saquinavir 24-hour AUC and Cmax (n=6) following the administration of a higher calorie meal were on average two times higher than after a lower calorie meal. The effect of food has been shown to persist for up to 2 hours. To attempt to minimize inter-subject variability and the food effect of saquinavir mesylate seen in man, an oral solid dosage form having a uniform and rapid dissolution profile of saquinavir mesylate would be desirable.

Particle size reduction is one means in which to minimize inter-subject variability and improving bioavailability. Micronization is an approach used to reduce particle size. Upon micronization, saquinavir mesylate has a tendency to agglomerate, however, thus reducing the surface area of its primary particles in contact with the dissolution medium. Micronized saquinavir mesylate exhibits a slow dissolution rate.

For adult patients, the recommended dose of INVIRASE® in combination with a nucleoside analogue is 3×200 mg capsules three times daily within 2 to 4 hours after food. In view of this dosage regime, patient compliance is a real concern. Treatment success could be improved by encouraging better adherence, as for example, by reducing the number of dosage units that must be taken per day. A unit dosage form containing a higher amount of saquinavir would thus be useful. The problem of drug agglomeration worsens, however, with increased drug loading of micronized saquinavir mesylate.

### SUMMARY OF THE INVENTION

The present invention provides a solid unit oral pharmaceutical dosage form of saquinavir mesylate comprising from about 60% to about 80% micronized saquinavir mesylate based on the mesylate salt, from about 4% to about 8% of a pharmaceutically acceptable water soluble binder, a pharmaceutically acceptable disintegrant, and a pharmaceutically acceptable carrier, wherein each percentage is of the kernel weight of the pharmaceutical dosage form. (The about 60% to about 80% micronized saquinavir mesylate corresponds to an amount of from about 200 mg to about 800 mg saquinavir mesylate calculated as saquinavir free base.)

The present invention provides a solid unit oral pharmaceutical dosage form of saquinavir mesylate comprising micronized saquinavir mesylate in an amount of from about 250 mg to about 800 mg calculated as free base, a pharmaceutically acceptable binder, a pharmaceutically acceptable disintegrant, and a pharmaceutically acceptable water soluble carrier.

### BRIEF DESCRIPTION OF THE DRAWINGS

FIG. 1 presents dissolution profiles of the invented formulation in a tablet dosage form, Example 1 (500 mg as free base), indicating lot-to-lot reproducibility

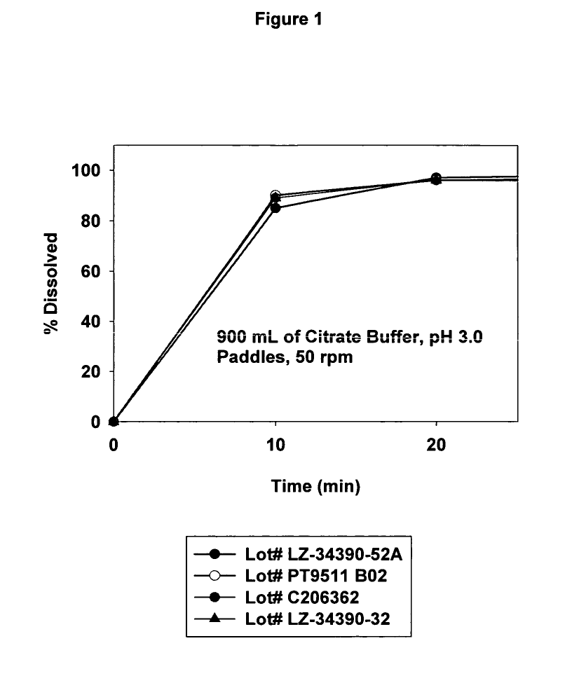

FIG. 2 presents dissolution profiles of the current market formulation in a capsule dosage form, Example 2 (200 mg as free base), indicating lot-to-lot variability.

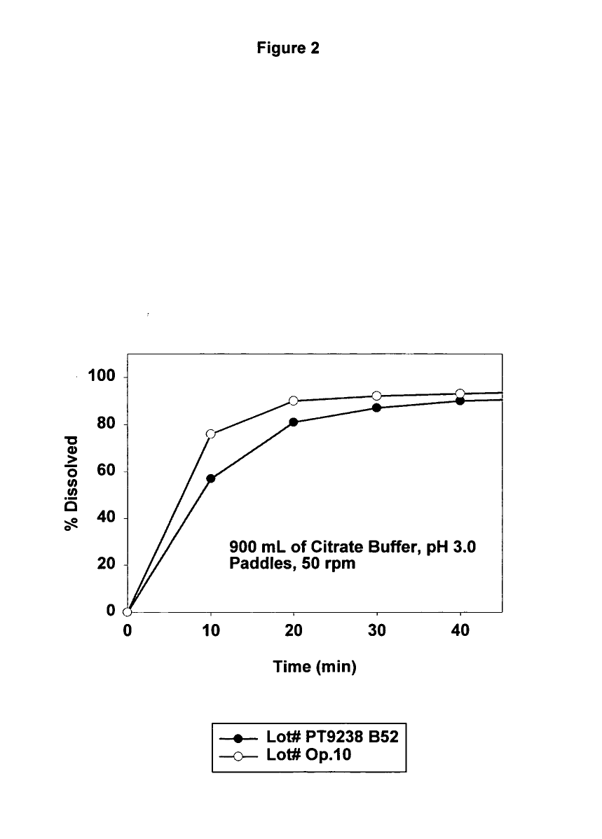

FIG. 3 presents dissolution profiles of the invented formulation in a tablet dosage form (Example 1) compared to the current market formulation in a capsule dosage form (Example 2) at a dose of 1000 mg saquinavir as free base

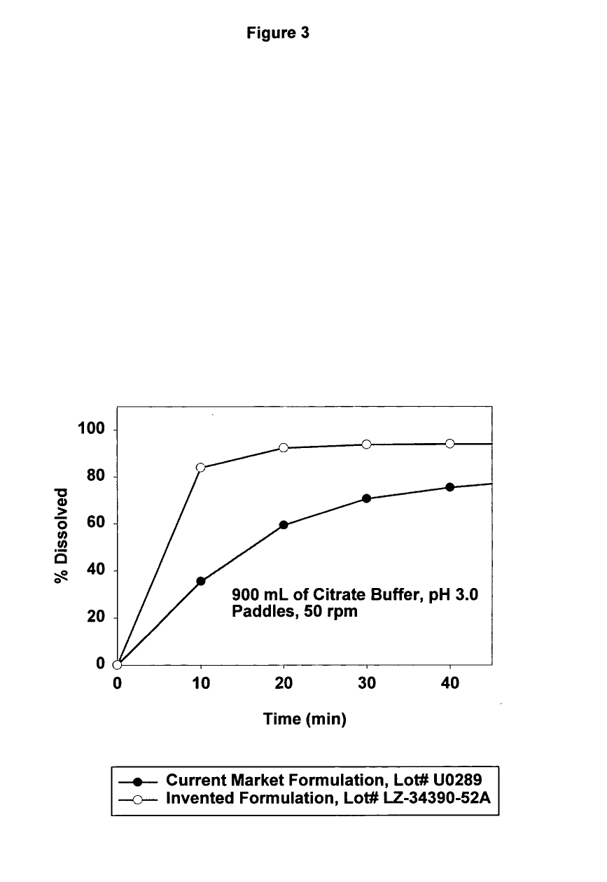

FIG. 4 presents dissolution profiles of the invented formulation in a capsule dosage form (Example 3) compared to the current market formulation in a capsule dosage form (Example 2) at a dose of 1000 mg saquinavir as free base

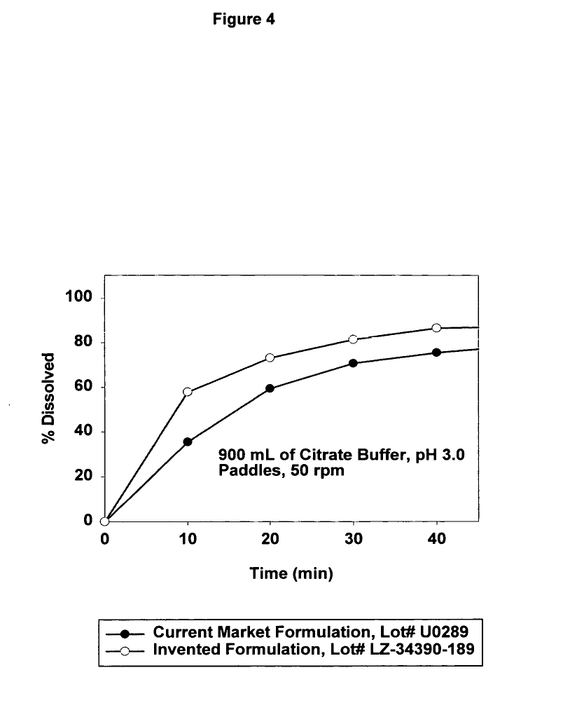

FIG. 5 presents dissolution profiles of the invented formulation in a tablet dosage form, Example 1 (500 mg as saquinavir free base), indicating a rapid and highly reproducible dissolution profile regardless of compression force applied

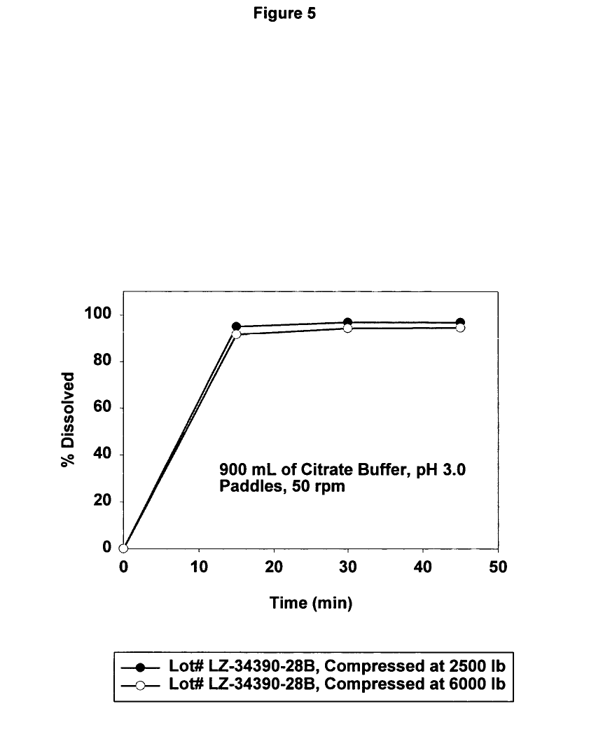

FIG. 6 presents dissolution profiles of the invented formulation in a tablet dosage form (Example 1) compared to a conventional tablet formulation (Example 4) at a dose of 1000 mg saquinavir as free base

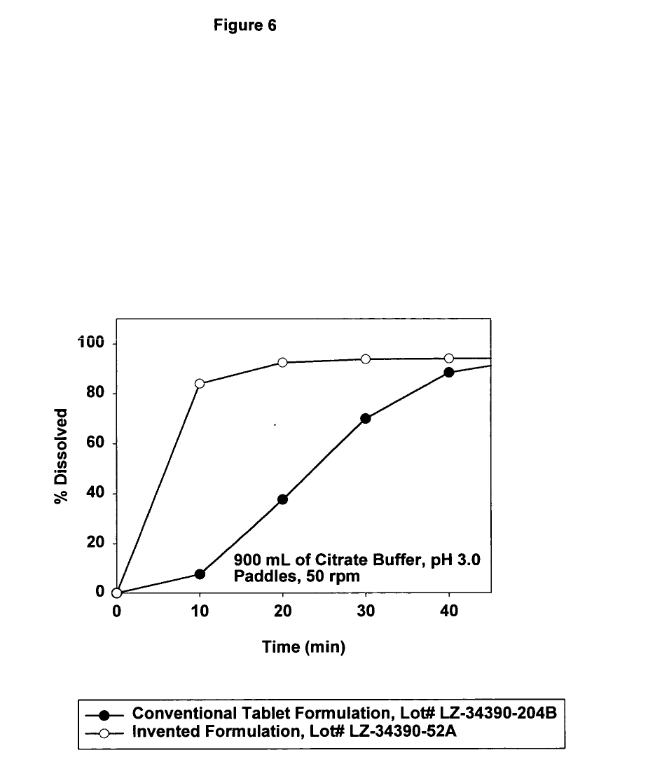

FIG. 7 presents dissolution profiles of the invented formulation in a tablet dosage form, Example 1 (500 mg as free base), indicating a rapid and highly reproducible dissolution profile regardless of the granulation end point

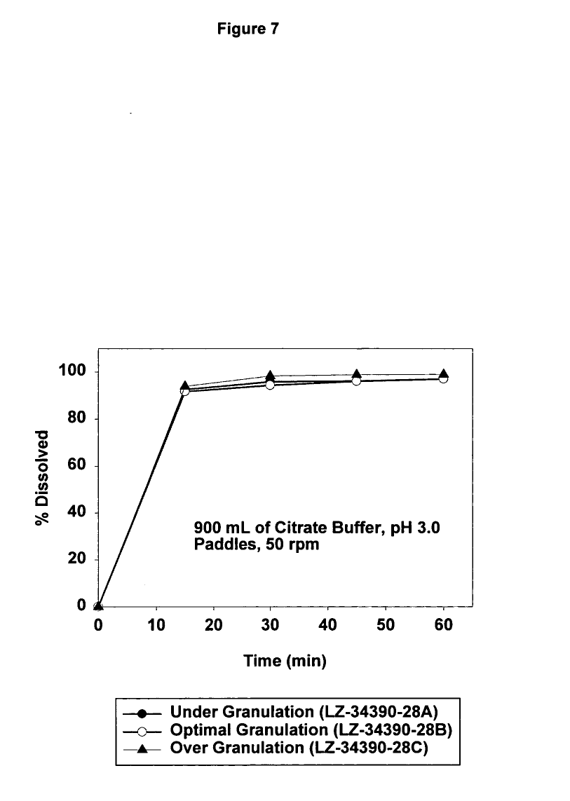

### DETAILED DESCRIPTION OF THE INVENTION

The pharmaceutical dosage form of micronized saquinavir mesylate in accordance with the present invention provides a rapid and highly reproducible dissolution profile. The saquinavir mesylate dosage form of the present invention may be used to treat HIV-infected individuals. Coadministration with another antiretroviral drug, for example, ritonavir, is contemplated.

The present invention provides a solid unit oral pharmaceutical dosage form of saquinavir mesylate comprising micronized saquinavir mesylate in an amount of from about 200 mg to about 800 mg calculated as saquinavir base and a pharmaceutically acceptable water soluble carrier, such as lactose monohydrate, present from about 3% to about 10% by weight of the kernel. A pharmaceutically acceptable water-soluble binder is present from about 4 to 8% by weight of the kernel. A pharmaceutically acceptable disintegrant is present from about 3 to 10% by weight of the kernel. A granulation of the saquinavir mesylate, the carrier, the binder, and at least a portion of the disintegrant is prepared. This granulation consists of various particle sizes of agglomerates of saquinavir mesylate, carrier, binder and disintegrant, and the resulting powder is free-flowing with advantageous compaction and wetting properties. When the unit dosage is a tablet, the tablet is prepared in part from these agglomerates. When the tablet is exposed to gastrointestinal fluids, it disintegrates and releases micronized saquinavir mesylate for rapid dissolution.

After preparing the granulation, microcrystalline cellulose (MCC) may be added as an extra-granular component to enhance mechanical strength of the produced tablets. MCC is present from about 5 to 20% by weight of the kernel. A lubricant such as magnesium stearate may be added as an extra-granular component, from about 0.5 to 1.2% by weight of the kernel.

The present invention also provides a process for preparing a solid unit oral pharmaceutical dosage form of saquinavir mesylate. The process involves micro-granulation of the drug with a disintegrant and a hydrophilic binder and carrier. The dosage form thus produced retains saquinavir mesylate in crystalline form.

The invented formulation, in either a tablet or capsule dosage form, exhibits a relatively faster and much more reproducible dissolution profile compared to the profile of the current market capsule formulation. Moreover, the oral dosage form disclosed herein provides a rapid and highly reproducible dissolution profile, irrespective of compression force and granulation end point. The dosage form of the present invention advantageously has a weight of from about 400 mg to about 1.5 g.

The solid unit oral pharmaceutical dosage form of the present invention contains a kernel and a kernel containing portion. The kernel comprises the saquinavir mesylate, binder, disintegrant, and carrier, in accordance with the present invention. The kernel optionally includes one or more pharmaceutically acceptable excipients, for example, lactose monohydrate. The kernel is preferably comprised of an admixture of a granulation and excipients added to the granulation (“extra-granulation”). The kernel containing portion may be, for example, a tablet film coating, or a capsule or caplet coating.

The saquinavir mesylate used in the present invention is micronized to small particle size. Micronized saquinavir mesylate is typically saquinavir mesylate having particles ranging from about 1 to about 20 microns. In preparing the solid unit oral pharmaceutical dosage form, micronized saquinavir mesylate calculated based on the mesylate salt is used in an amount such that it is about 60% to about 80% of the kernel weight. This corresponds to an amount of micronized saquinavir mesylate calculated as saquinavir free base of from about 200 mg to about 800 mg.

Pharmaceutically acceptable excipients which may optionally be used in the present invention include types of excipients other than those recited. Thus, the percentage of binder and disintegrant in the kernel, for example, remain as indicated. Pharmaceutically acceptable excipients that may optionally be used include microcrystalline cellulose and lubricants.

Lubricants include, for example, magnesium stearate and talc. Magnesium stearate is preferred. A lubricant can be used as an extra-granulation ingredient of the kernel. A lubricant is preferably present from 0.5% to 1.2% by weight of the kernel.

The excipient added to the milled, dried granulation can be selected from the group of lubricants, disintegrants and diluents. The pharmaceutical excipient may be, for example, microcrystalline cellulose, corn starch, magnesium stearate, etc. For preparing tablets, the solid dosage form can be processed into a solid unit oral dosage form by granulating, milling, blending, lubricating, compressing (tabletting), and, typically, aqueous film coating.

The pharmaceutical dosage form of the present invention is prepared by micro-granulating micronized saquinavir mesylate with the disintegrant and the hydrophilic binder and carrier, and milling. Preferably, the granulation is blended with lubricant, tabletted and aqueous-based film coated. During the micro-granulation process, micronized saquinavir mesylate is deagglomerated and wetted by the hydrophilic carrier and binder, thereby maximizing the surface area of its primary particles in contact with the dissolution medium.

A method for preparing a solid unit oral pharmaceutical dosage form of micronized saquinavir mesylate is provided, comprising spraying a solution of water soluble binder on an admixture of from about 200 mg to about 800 mg micronized saquinavir mesylate calculated as free base, a pharmaceutically acceptable disintegrant, and a pharmaceutically acceptable water soluble carrier, to achieve a uniform granulation of the present invention. Preferably, the carrier is lactose monohydrate, and the disintegrant is croscarmellose sodium.

Preferably, a portion of the disintegrant is included in the granulation, and the remaining portion of the disintegrant is added as an extra-granular component, and blended. The ratio of disintegrant in the granulation to disintegrant in the extra-granulation is from about 3:1 to about 1:1. Preferably, the ratio is from about 2.5:1 to about 1.5:1. More preferably, the ratio is about 2:1.

Preferably, microcrystalline cellulose is added as an extra-granular component and blended with the granulation to enhance mechanical strength of the resulting tablets. Microcrystalline cellulose is present from about 5% to about 20% by weight of the kernel, preferably from about 5% to about 15%.

Preferably, a lubricant such as magnesium stearate, is added externally to the granulation to provide adequate lubricity to tablet punch tooling during compression. The lubricant is present from about 0.5% to about 1.2% by weight of the kernel.

In an embodiment of the present invention, tablets are prepared as follows:
a) Blend micronized saquinavir mesylate in an amount of from about 200 mg to about 800 mg (calculated as free base) per unit dosage with a hydrophilic carrier and disintegrant in a high shear granulator.
b) Spray or slowly add an aqueous binder solution in water to the powder mix from step (a) while mixing to achieve the optimal granulation end point.
c) De-lump the wet granulation from step (b) into a uniform granulation.
d) Dry the wet granulation from step (c) in a forced-air oven set at 40°-50° C. or in a fluid bed dryer with inlet air temperature of 50°-60° C. until the moisture content of the granulation is in the range of 1.5-2%.
e) Mill the dried granulation from step (d).
f) Blend the milled granulation from step (e) with other suitable tablet diluents, such as microcrystalline cellulose, and disintegrant.
g) Lubricate the blend from step (f) with a suitable lubricant, such as magnesium stearate.
h) Compress the final blend from step (g) on a tablet press.
i) Aqueous film coat the tablet from step (h).

### EXAMPLES

In the following Examples, the optimal granulation end point was determined by visual inspection as the point at which the granulation had no further detectable change in particle size.

### Example 1

TABLE 1
500 mg Saquinavir Mesylate Tablet (Invented Formulation)

| Ingredients | mg/tablet |
|---|---|
| Micronized Saquinavir Mesylate | 571.50* |
| Lactose Monohydrate | 38.50 |
| Croscarmellose Sodium | 45.00 |
| Povidone K30 | 40.00 |
| Microcrystalline Cellulose (Avicel PH 101) | 95.00 |
| Magnesium Stearate | 10.00 |
| KERNEL WEIGHT | 800.00 |
| Hydroxypropyl Methylcellulose 2910 (6 cps) | 7.01 |
| Titanium Dioxide | 4.32 |
| Talcum | 4.32 |
| Iron Oxide Yellow | 0.57 |
| Iron Oxide Red | 0.08 |
| Ethylcellulose Dispersion (solids) | 2.32 |
| Triacetin | 1.38 |
| TOTAL WEIGHT | 820.00 |

*Equivalent to 500 mg as free base

### I. Preparation of Kernels

A. Micronized saquinavir mesylate, lactose monohydrate and a portion of croscarmellose sodium (approximately 66.7% of total amount of croscarmellose sodium) were mixed in a high shear granulator for 5 minutes using impeller at low speed and agitator at low speed.

B. Preparation of 20% w/w Povidone K30 Solution: In a stainless steel container, Povidone K30 was slowly added to Purified Water (160 mg/tablet) and mixed using a propeller mixer. Mixing was continued until Povidone K30 was completely dissolved.

C(1). The powder mix from Step A was granulated by spraying the 20% w/w Povidone K30 Solution from Step B onto the powder mix in the high shear granulator and continually mixed using impeller at low speed and agitator at low speed for over 8-10 minutes.

C(2). Additional Purified Water (approximately 180 mg/tablet) was sprayed onto the powder mix from Step C(1) which was continually mixed using impeller at low speed and agitator at low speed for over 8-10 minutes. Additional kneading of the granulation was performed to achieve an optimal granulation end point. The wet granulation was discharged with impeller at low speed and agitator at low speed into a polyethylene-lined container.

D. The wet granulation was delumped by passing through a Co-mil equipped with a 19.05-mm round opening screen (#750Q) at 1350 rpm or through a Frewitt rotating sieve equipped with a 10-mm round opening screen at 1000-2000 rpm.

E. The delumped wet granulation from Step D was dried in a fluid bed dryer with inlet air temperature set at 65°±10° C. until the moisture content of the granulation, determined by loss on drying using an Omnimark Moisture Analyzer set at 90° C., was less than 1.8%.

F. The dried granulation from Step E was milled through a Co-mil equipped with a 1.27-mm round, grated opening screen (#050G) at 4500 rpm or through a Frewitt hammer mill equipped with a 2.0-mm round screen using knives forward at 3170 rpm.

G. The milled granulation from Step F, Avicel PH 101 and the remainder of croscarmellose sodium (approximately 33.3% of total croscarmellose sodium) were mixed in a PK Blender or equivalent for 10 minutes.

H. Approximately 50% of the granulation from the PK Blender in Step G was removed.

I. Magnesium Stearate (passed through a #30 mesh stainless steel screen) was added to the PK Blender from Step H. The removed granulation from Step H was placed back into the PK Blender and mixed for 5 minutes.

J. The granulation from Step I was compressed using the following specifications:
Punch Size: Oval-shaped, 8.74 mm×18.75 mm, standard concave
Tablet Weight: 800 mg (760-840 mg)
Tablet Hardness: 30 SCU (25-35 SCU) or 210 N (175-245 N)

### II. Application of the Film Coat

A. Preparation of the Film Coating Suspension

In a stainless steel container, Triacetin and Aquacoat ECD-30 were dispersed in Purified Water using a propeller mixer and mixed for 45 minutes.

A powder mixture of hydroxypropyl methylcellulose 2910 (6 cps), talc, titanium dioxide, yellow iron oxide and red iron oxide was added to the dispersion, which was mixed gently to avoid air entrapment. Mixing was continued for another 60 minutes or until a uniform suspension was obtained.

B Application of the Film Coat

The kernels from Step J of Section I (PREPARATION OF KERNELS) were placed into a perforated coating pan. The inlet temperature was slowly increased to 60°±10° C. to warm the kernels, with intermittent jogging, until the outlet temperature reached 40°±5° C.

With the inlet air temperature at 60°±10° C., the pan speed was increased to provide sufficient rotation of the kernels inside the pan. The kernels were sprayed with the Film Coating Suspension from Section IIA above and stirred continuously using an air spray system. The product temperature was maintained at 45°±5° C. 20 mg of the film coat (range 17-23 mg) was applied on a dry basis per tablet.

The inlet air temperature was reduced to 50°±5° C. and the pan speed to 4±2 rpm. Drying of the coated tablets was continued for 2-4 minutes.

The inlet air temperature was reduced to 40°±5° C. and the coated tablets were dried by jogging until the moisture content of the tablets, determined by loss on drying using an Omnimark Moisture Analyzer set at 90° C., was less than 2.0%. The heat was turned off and the tablets cooled to room temperature by occasional jogging.

### Example 2

TABLE 2
200 mg Saquinavir Mesylate Capsule (Market Formulation)

| Ingredients | mg/capsule |
|---|---|
| Micronized Saquinavir Mesylate | 228.70* |
| Sodium Starch Glycolate | 16.00 |
| Lactose Anhydrous | 63.30 |
| Microcrystalline Cellulose (Avicel PH 102) | 60.00 |
| Povidone K30 | 8.00 |
| Magnesium Stearate | 4.00 |
| Talcum | 28.00 |
| FILL WEIGHT | 408.00 |
| Capsule Shell (Size #0) | 96.00 |
| TOTAL WEIGHT | 504.00 |

*Equivalent to 200 mg as free base

A. Micronized saquinavir mesylate, lactose anhydrous, microcrystalline cellulose and a portion of sodium starch glycolate (56.25% of total amount of sodium starch glycolate) were mixed in a high shear granulator.

B. Povidone K30 Solution was added to the powder mix in the high shear granulator (Step A) and continually mixed, to granulate the powder mix.

C. Additional Purified Water was added to the powder mix from Step B, which was continually mixed until an optimal granulation end point was obtained. The wet granulation was discharged into a polyethylene-lined container.

D. The wet granulation from Step C was delumped through a mill.

E. The delumped wet granulation from Step D was dried in a fluid bed dryer with inlet air temperature set at 65°±10° C. until the moisture content of the granulation, determined by loss on drying using an Omnimark Moisture Analyzer set at 90° C., was less than 1.8%.

F. The dried granulation from Step E was passed through a mill.

G. The milled granulation from Step F was mixed with a portion of sodium starch glycolate (43.75% of total amount of sodium glycolate, talc and magnesium stearate) in a blender.

H. The final blend from Step G was encapsulated in a capsule (#0) at a target fill weight of 408 mg using a capsule filling machine.

### Example 3

TABLE 3
200 mg Saquinavir Mesylate Capsule (Invented Formulation)

| Ingredients | Mg/capsule |
|---|---|
| Micronized Saquinavir Mesylate | 228.70* |
| Lactose Monohydrate | 15.30 |
| Sodium Croscarmellose | 18.00 |
| Povidone K30 | 16.00 |
| Microcrystalline Cellulose (Avicel PH 101) | 38.00 |
| Magnesium Stearate | 4.00 |
| FILL WEIGHT | 320.00 |
| Capsule Shell (Size #0) | 96.00 |
| TOTAL WEIGHT | 416.00 |

*Equivalent to 200 mg as free base

For Steps A to I, follow the same procedure specified in Example 1.

Step J. The final blend from Step I was encapsulated in a capsule (#0) at a target fill weight of 320 mg using a capsule filling machine.

### Example 4

TABLE 4
500 mg Saquinavir Mesylate Tablet Produced by Prior
Methodology

| Ingredients | Mg/tablet |
|---|---|
| Micronized Saquinavir Mesylate | 571.50* |
| Povidone K30 | 20.00 |
| Lactose Anhydrous | 158.25 |
| Microcrystalline Cellulose (Avicel PH 102) | 150.00 |
| Sodium Starch Glycolate | 40.00 |
| Talcum | 70.00 |
| Magnesium Stearate | 10.00 |
| TOTAL TABLET WEIGHT | 1019.75 |

*Equivalent to 500 mg free base

For Steps A to G, follow the same procedures specified in Example 2.

Step H. The granulation from Step G was compressed using the following specifications:
Punch Size: Oval-shaped, 9.28 mm×20.02 mm, standard concave
Tablet Weight: 1200 mg (1140-1260 mg)
Tablet Hardness: 25-35 SCU or 175-245 N
   Dissolution Testing

Oral dosage forms containing saquinavir mesylate (Examples 1-4) were evaluated for dissolution in 900 mL of citrate buffer, pH 3.0, equilibrated at 37°±0.5° C. using a paddle method (USP Apparatus 2) at 50 rpm. Sample aliquots were taken at different time intervals and analyzed by UV spectrophotometry.

1. A solid unit oral pharmaceutical dosage form of saquinavir mesylate comprising from about 60% to about 80% micronized saquinavir mesylate based on the mesylate salt, from about 4% to about 8% of a pharmaceutically acceptable water soluble binder, a pharmaceutically acceptable disintegrant, and a pharmaceutically acceptable carrier, wherein each percentage is of the kernel weight of the pharmaceutical dosage form.

2. The dosage form according to claim 1, wherein the saquinavir mesylate is from about 70% to about 75% of the kernel weight of the pharmaceutical dosage form.

3. The dosage form according to claim 1, wherein the saquinavir mesylate in the pharmaceutical dosage form is in an amount of from about 200 mg to about 700 mg calculated as saquinavir free base.

4. The dosage form according to claim 3, wherein the saquinavir mesylate in the pharmaceutical dosage form is in an amount of from about 250 mg to about 700 mg calculated as saquinavir free base.

5. The dosage form according to claim 4, wherein the saquinavir mesylate in the pharmaceutical dosage form is in an amount of about 500 mg calculated as saquinavir free base.

6. The dosage form according to claim 1, wherein the carrier is water soluble.

7. The dosage form according to claim 1, wherein the carrier is present from about 3% to about 10% of the kernel weight.

8. The dosage form according to claim 7, wherein the carrier is present from about 4% to about 6% of the kernel weight.

9. The dosage form according to claim 1, wherein the water soluble carrier has a particle size of from about 30 microns to about 200 microns.

10. The dosage form according to claim 1, wherein the water soluble carrier is lactose monohydrate.

11. The dosage form according to claim 10, wherein the lactose monohydrate has a particle size of from about 100 microns to about 150 microns.

12. The dosage form according to claim 1, wherein the binder is present from about 4% to about 6% of the kernel weight.

13. The dosage form according to claim 1, wherein the binder is polyvinylpyrrolidone.

14. The dosage form according to claim 1, wherein the disintegrant is present from about 3% to about 10% of the kernel weight.

15. The dosage form according to claim 14, wherein the disintegrant is present from about 4% to about 8% of the kernel weight.

16. The dosage form according to claim 1, wherein the disintegrant is selected from croscarmellose sodium and crospovidone.

17. The dosage form according to claim 1, further comprising microcrystalline cellulose.

18. The dosage form according to claim 17, wherein the microcrystalline cellulose is present from about 5% to about 20% of the kernel weight.

19. The dosage form according to claim 18, wherein the microcrystalline cellulose is present from about 5% to about 15% of the kernel weight.

20. The dosage form according to claim 1, wherein the dosage form further comprises a lubricant.

21. The dosage form according to claim 1, wherein the kernel of the pharmaceutical dosage form consists of an admixture of a granulation and an extra-granulation, wherein said granulation comprises the micronized saquinavir mesylate, the binder, the carrier, and disintegrant, and the extra-granulation comprises disintegrant, wherein the ratio of disintegrant in the granulation to disintegrant in the extra-granulation is from about 3:1 to about 1:1.

22. The dosage form according to claim 21, wherein the ratio is about 2.5:1 to about 1.5:1.

23. The dosage form according to claim 22, wherein the ratio is about 2:1.

24. The dosage form according to claim 21, further comprising microcrystalline cellullose, which is comprised in the extra-granulation of the kernel.

25. The dosage form according to claim 21, further comprising a lubricant, which is comprised in the extra-granulation of the kernel.

26. The dosage form according to claim 1, which is selected from the group consisting of a tablet, a capsule and a caplet.

27. The dosage form according to claim 1, having a weight of from about 400 mg to about 1.5 g.

28. A solid unit oral pharmaceutical dosage form of saquinavir mesylate comprising micronized saquinavir mesylate in an amount of from about 250 mg to about 800 mg calculated as free base, a pharmaceutically acceptable binder, a pharmaceutically acceptable disintegrant, and a pharmaceutically acceptable water soluble carrier.

29. The solid unit oral pharmaceutical dosage form according to claim 28 in which the micronized saquinavir mesylate is in an amount of about 500 mg.

30. The solid unit oral pharmaceutical dosage from according to claim 1, wherein the saquinavir mesylate is in crystalline form.

31. The solid unit oral pharmaceutical dosage form according to claim 1, which is prepared by micro-granulation of the saquinavir, the binder, the carrier, and at least a portion of the total amount of the disintegrant contained in the dosage form.
